(19) United States
(12) Patent Application Publication
(10) Pub. No.: US 2004/0166162 A1
(43) Pub. Date: Aug. 26, 2004

(54) Novel pharmaceutical formulation containing a proton pump inhibitor and an antacid

(76) Inventors: Robert Niecestro, Pocono Pines, PA (US); Unchalee Kositprapa, Davie, FL (US); Yoon Oh, Pembroke Pines, FL (US); Avinash Nangia, Weston, FL (US); John R. Cardinal, Tamarac, FL (US); Elliot F. Hahn, North Miami Beach, FL (US)

Correspondence Address: Martin P. Endres, Esq., HEDMAN & COSTIGAN, P.C., 1185 Avenue of the Americas, New York, NY, 10036, US

(21) Appl. No.: 10/761,805

(22) Filed: Jan. 21, 2004

Related U.S. Application Data
(60) Provisional application No. 60/442,337, filed on Jan. 24, 2003.

(51) Int. Cl. A61K 31/4439; A61K 9/24

(52) U.S. Cl. 424/472; 514/339

(57) ABSTRACT

The subject invention is multi layer pharmaceutical dosage form comprising at least two layers whereby a proton pump inhibitor is in one distinct layer and an aluminum, magnesium or calcium antacid salt is in a second distinct layer.

This application claims the benefit of U.S. provisional application Serial No. 60/442,337 filed Jan. 24, 2003.

### SUMMARY OF THE INVENTION

The present invention relates to a pharmaceutical dosage form comprising a proton pump inhibitor, in combination with an antacid. More particularly, the present invention relates to a multiple layer pharmaceutical dosage form whereby a proton pump inhibitor is in one distinct layer and an aluminum, magnesium or calcium antacid salt is in second or third distinct layers. The multi layer arrangement can be in the form of a compressed tablet or a filled gelatin capsule.

### BACKGROUND OF THE INVENTION

Many techniques have been used to provide proton pump inhibitor (PPI) therapy. Included in these techniques are delayed, controlled and extended-release pharmaceutical dosage forms.

The key action mechanism of the PPIs is inhibition of H+/K+-adenosine triphosphate (also known as acid pump or proton pump), an enzyme present in the gastric parietal cells. It is believed that these drugs are metabolized in the parietal cells to active sulfenamide metabolites that inactivate the sulfhydryl group of the proton pump, thus reducing the hydrogen ion secretion. PPIs are generally lipophilic weak bases with poor aqueous solubility at low pH. Many PPIs are unstable in low pH solutions and undergo rapid acid-catalyzed degradation, and they are relatively stable at neutral or high pH.

Due to the pH sensitivity of PPIs, effective drug delivery is problematic, as the pH of the gastric environment is acidic and the pH of the intestinal region is relatively alkaline. For the drug to be therapeutically active after oral administration, it should be protected from the acid present in the gastric juices. Further, the drug should reach the upper small intestinal region in an intact, absorbable form, where the drug can be rapidly absorbed to reduce acid production.

Enteric coating is by far the most popular method of protecting an acid-labile drug from gastric degradation. In this method, either the drug particles or the dosage form is coated with a polymer that does not dissolve in the low pH gastric environment, but dissolves in the alkaline environment of the small intestine. Currently, PPIs are administered as enteric-coated solid dosage forms. Typically, these enteric coats dissolve at a pH of approximately 5.5 or greater.

Tabletted effervescent dosage forms of enteric-coated proton pump inhibitors including sodium carbonate and bicarbonate are disclosed in WO 97/25030 and U.S. Pat. No. 6,132,770. In addition, U.S. Pat. No. 5,840,737 discloses a pharmaceutical composition including an aqueous solution/suspension of omeprazole or other substituted benzimidazoles in a carrier including a bicarbonate salt of a Group IA metal.

However, there are some problems associated with enteric-coated preparations. These preparations are difficult to formulate as liquids, which may inconvenience pediatric patients or a patient population that has difficulty in swallowing. Moreover, the enteric coating must dissolve before the drug may be available for absorption. Since dissolution of the enteric coating is pH-dependent, and the pH profile of the gastrointestinal tract in an individual is variable at different times and is dependent on numerous physiological factors (e.g., the fed or fasted state), variable dissolution times for the enteric coat and variable pharmacokinetic profiles in individuals may result.

The acid-labile drugs for oral administration may also be protected from gastric acidity by neutralizing the pH of the gastric fluid. Such a technique is described in an article by Pilbrant and Cederberg entitled: “Development of an Oral Formulation of Omeprazole”, Scand. J. Gastroenterology, 1985, Suppl. 108, pp. 113-120. Some formulations incorporate an acid neutralizer and enteric-coated PPI to create a stable formulation such as WO 94/02140, which discloses a core, composed of an antacid combination and U.S. Pat. No. 6,096,340 which discloses an enteric-coated formulation containing omeprazole, a surface-active agent, a filler, a pharmaceutically acceptable alkaline agent and a binder.

Co-administration of enteric-coated omeprazole, with 8.4% sodium bicarbonate solution/suspension via the nasogastric tube, has been disclosed by Phillips et al. in “A Prospective Study of Simplified Omeprazole Suspension for the Prophylaxis of Stress-Related Mucosal Damage”, Crit. Care Med, 1996, Vol. 24, No. 11, and Sharma et al. in “The Effects on Intragastric Acidity of Per-Gastronomy Administration of an Alkaline Suspension of Omeprazole”, Aliment Pharmacol. Ther., 13:1091-1095 (1999). Before administering, the enteric-coated drug granules were shaken with the sodium bicarbonate solution for a sufficient time period until a milky white suspension resulted, to dissolve the enteric coating in the sodium bicarbonate solution. A large quantity of sodium bicarbonate must be administered with each dose of omeprazole, in the method described above. However, there is a major disadvantage in using large quantities of sodium bicarbonate orally, since sodium bicarbonate, upon neutralization in the gastric fluid, produces gases and results in belching (see e.g. U.S. Pat. No. 5,840,737). This is detrimental to patients suffering from gastro-esophageal reflux disease (GERD).

In an attempt to reduce the amount of co-administered sodium bicarbonate, Phillips et al. (WO 00/26185) found that only 10 milliliters of an 8.4% sodium bicarbonate solution were sufficient to provide effective acid neutralization and protect the enteric-coated omeprazole from degradation in the gastric environment. However, there is still a need for a method of PPI administration that is even more effective. In particular, a method that avoids the difficulties associated with the enteric-coating, yet still provides sufficient stability for either solid or liquid formulations would be particularly advantageous.

U.S. Pat. No. 6,183,776 describes a dosage form comprising a proton pump inhibitor and an antacid; however, it also requires the use of an enteric coating on the proton pump inhibitor.

There is a need to provide a combination dosage form comprising a proton pump inhibitor and an antacid that is chewable or rapidly dissolves in the oral cavity, palatable, and relatively easy to manufacture.

### SUMMARY OF THE INVENTION

The subject invention is a novel dosage form comprising a proton pump inhibitor and a calcium, magnesium or aluminum antacid in a dosage form comprising at least 2 layers wherein the proton pump inhibitor and antacid are each in distinct layers.

It is an object of the present invention to provide a multi layer dosage form combining a proton pump inhibitor, free of enteric coating, in one distinct layer, with an aluminum, magnesium or calcium salt of an antacid in a second distinct layer which provides antacid in sufficient amount to neutralize the gastric environment so as not to cause degradation of the non-enterically coated proton pump inhibitor.

It is another object of the present invention to provide a dosage form combining a proton pump inhibitor, free of enteric coating, in one distinct layer, with an aluminum, magnesium or calcium salt of an antacid in a separate and distinct layer, wherein the proton pump inhibitor and antacid are not in the same layer and where the dosage form comprises at least two layers.

It is another object of the present invention to provide a dosage form combining a proton pump inhibitor free of enteric coating, in one distinct layer, with an aluminum, magnesium or calcium salt of an antacid in a second separate and distinct layer, which provides immediate release of the proton pump inhibitor.

It is another object of the present invention to provide a dosage form combining a proton pump inhibitor free of enteric coating, in one distinct layer, with an aluminum, magnesium or calcium salt of an antacid in a second separate and distinct layer, in which the proton pump inhibitor is free from any enteric coating.

It is another object of the present invention to provide a dosage form combining a proton pump inhibitor free of enteric coating, in one distinct layer, with an aluminum, magnesium or calcium salt of an antacid in a second separate and distinct layer, that is easy to swallow.

It is another object of the present invention to provide a dosage form combining a proton pump inhibitor free of enteric coating, in one distinct layer, with an aluminum, magnesium or calcium salt of an antacid in a second separate and distinct layer, that is chewable.

It is another object of the present invention to provide a dosage form combining a proton pump inhibitor free of enteric coating, in one distinct layer, with an aluminum, magnesium or calcium salt of an antacid in a second separate and distinct layer, that rapidly dissolves in the oral cavity or mouth.

It is another object of the present invention to provide a dosage form combining a proton pump inhibitor free of enteric coating, in one distinct layer, with an aluminum, magnesium or calcium salt of an antacid in a second separate and distinct layer, that has taste masking ingredients to provide a chewable or rapidly dissolving dosage form substantially free of bitterness.

Finally, it is another object of the present invention to provide a dosage form combining a proton pump inhibitor with an aluminum, magnesium or calcium salt of an antacid in which there is an effective amount of proton pump inhibitor to treat ulcers and related disorders, and an effective amount of antacid to provide rapid and sustained relief from common heartburn for 24, 48 or 72 hours.

### DETAILED DESCRIPTION OF THE INVENTION

The present invention attempts to solve the forgoing objectives. The present invention is a multi-layered oral pharmaceutical dosage form that comprises at least one proton pump inhibitor layer and at least one antacid layer. In a preferred embodiment the antacid layer and the entire dosage form is free of sodium bicarbonate and any other effervescent materials. Also the entire dosage form is free of any enteric coatings. The dosage form may be in the form of a multi-layered compressed tablet or a multi-layered filled gelatin capsule. In a preferred embodiment the dosage form is chewable or rapidly disintegrating.

As used in this application the terms “rapidly disintegrating” and/or “rapidly dissolving” mean that the time for disintegration of the dosage form is generally less than one minute, preferably less than 40 seconds and most preferably less than 30 seconds when tested according to the procedures described in USP 26 test method <701> using deionized water as the medium.

The proton pump inhibitor layer may be combined with pharmaceutically acceptable excipients. These excipients may include, but would not be limited to: an alkaline agent, prefereably an alkaline amino acid, a filler, a disintegrant and a binder. The proton pump inhibitor and the selected excipients can be mixed with a solvent to form granule. Granules are prepared using pharmaceutically acceptable methods commonly known in the art. These methods may include, but would not be limited to, fluid bed granulation, granulation in a high shear granulator, granulation in a V-blender and roller compaction.

Proton pump inhibitors may include substituted benzimidazoles such as omeprazole, lansoprazole, pantoprazole, pariprazole, leminoprazole, and salts, isomers, and derivatives thereof.

Antacids that may be used in the antacid layer of the present invention include aluminum, magnesium and calcium salts of hydroxides, carbonates, sulfates, bicarbonates, silicates or other pharmaceutically acceptable antacid aluminum or calcium salts. Examples of some of the preferred antacid salts are magnesium hydroxide, magnesium carbonate, magnesium trisilicate, aluminum hydroxide, aluminum carbonate, calcium carbonate and combinations of the foregoing. Some possible combinations include aluminum hydroxide and magnesium hydroxide, aluminum hydroxide and magnesium trisilicate, calcium carbonate and magnesium hydroxide and aluminium hydroxide, magnesium hydroxide and calcium carbonate. The preferred antacids for use in the present invention are aluminum and calcium salts. The foregoing antacids are merely examples of acceptable antacids. Other antacids are known to those skilled in the art and can be found in standard reference literature such as Remington, the Science and Practice of Pharmacy 20^{th} Ed. and the United States Pharmacopeia (USP 26) which are incorporated herein by reference.

The pharmaceutically acceptable excipients such as binders, fillers, lubricants, glidants, disintegrants and taste masking agents which are combined with the proton pump inhibitor and antacid are commonly known in the art. Many of these pharmaceutically acceptable excipients are described in the Handbook of Pharmaceutical Excipients 4^{th} Ed., Remington, the Science and Practice of Pharmacy 20^{th} Ed. and the United States Pharmacopeia (USP 26) which are incorporated herein by reference.

If a binder is used in the present invention, it may be any pharmaceutically acceptable, non-toxic pharmaceutically acceptable binder. The binder may be a water-soluble polymer of the group consisting of polyvinyl alcohol, polyvinylpyrrolidone, methylcellulose, hydroxypropyl cellulose, hydroxymethyl cellulose, hydroxy propyl methyl cellulose, hydroxyethyl methyl cellulose, gelatin, pectin, carrageenan, compressible sugars, sodium carboxymethyl cellulose, liquid glucose, alginates and gums and the like. The binder may also be a water insoluble binder such as ethylcellulose, acrylic or methacrylic polymers or copolymers, tragacanth, starch and pregelatinized starch and the like.

If a filler is used in the present invention, it may be any pharmaceutically acceptable, non-toxic pharmaceutically acceptable binder. The most common fillers are sugars such as lactose, dextrose, sucrose, maltose, mannitol, sorbitol, dibasic calcium phosphate and various starches or microcrystalline cellulose.

Examples of disintegrants that can be used in the present invention are corn starch, croscarmelose sodium, crospovidone (polyplasdone XL-10), sodium starch glycolate (EXPLOTAB or PRIMOJEL) or any combination of the foregoing. The most preferred disintegrant is crospovidone or sodium starch glycolate.

Taste masking agents are required for the chewable and rapidly disintegranting dosage forms of the present invention and include artificial sweeteners such as aspartame, saccharin, dipotassium glycyrrhizinate, stevia, thaumatin and flavorants such as citric acid, peppermint oil, wintergreen oil, menthol, lemon, lime, orange grape, cherry and vanilla extract. Additional taste masking agents are described in U.S. Pat. No. 6,027,746 and Vol. 1, pages 306-309 of Pharmaceutical Dosage Forms (Tablets) by Lieberman and Lachman, ©1982 which are incorporated herein by reference. In a preferred embodiment of the present invention, the taste masking agent comprises a mixture of artificial sweeteners and flavorants such as aspartame and peppermint oil or grape extract.

An alkaline agent may be necessary to stabilize the proton pump inhibitor during manufacture and storage of the dosage form. The alkaline agent can be any type of alkaline agent such as amino acids such as lysine, arginine, omitine, histidine, organic buffering compounds such as tromethamine, N-amino sugars, such as meglumine, eglumine, glucosamine, heterocyclic amine derivatives such as piperazine, alkali salts of citric acid, tartaric acid, caproic acid or fatty acids, alkali metal phosphates, silicates, hydroxides or carbonates, organic amines such as ethylamine, alkaline ammonium salts and combinations of the foregoing. Additional examples of alkaline agents can be found in U.S. Pat. No. 6,013,281 which are incorporated herein by reference. The preferred alkaline agents are amino acids such as arginine, lysine or meglumine.

The present invention may also comprise conventional processing aids such as tablet lubricants (magnesium stearate, sodium stearate), glidants (colloidal silicon dioxide) and wetting agents or stabilizers and surfactants (sodium lauryl sulfate, polysorbates). The processing aids are generally added to the dosage formulation in small amounts (less than 5 weight percent of the total weight of the formulation) and do not materially affect the properties of the final dosage formulation. Some of the aforementioned excipients can perform more than one function in the formulation. For example, sucrose and lactose can serve as fillers and sweeteners and microcrystalline cellulose can serve as a filler and a disintegrant depending upon the amount and manner used. The multi-function excipients are known to those skilled in the art.

The combination may comprise components in many different dosage strengths. Some examples of dosage strengths are herein provided, the strengths are meant by way of example and are in no way intended to be limiting or encompassing.

The antacid should be sufficient to neutralize the acid in the stomach and allow the proton pump inhibitors to be absorbed in the stomach and/or pass through the stomach relatively intact. Proton pump inhibitors are acid liable and therefore the acid in the stomach must be present in a sufficient amount to neutralize acid in order to protect the combination product. The neutralization of the stomach acid will also provide the added benefit of immediate relief for a patient until the proton pump inhibitor can begin working. Examples of embodiments of the subject invention may include:

Omeprazole (≧1 mg) with antacid (to produce ≧1 mEq of acid neutralizing capacity (ANC)).

Lansoprazole (≧1 mg) with antacid (to produce ≧1 mEq of acid neutralizing capacity (ANC)).

Rabeprazole (≧1 mg) with antacid (to produce ≧1 mEq of acid neutralizing capacity (ANC)).

Pantoprazole (≧1 mg) with antacid (to produce ≧1 mEq of acid neutralizing capacity (ANC)).

Esomeprazole (≧1 mg) with antacid (to produce ≧1 mEq of acid neutralizing capacity (ANC)).

Pariprazole (≧1 mg) with antacid (to produce ≧1 mEq of acid neutralizing capacity (ANC)).

Leminoprazole (≧1 mg) with antacid (to produce ≧1 mEq of acid neutralizing capacity (ANC)).

Omeprazole (≧1 mg) with antacid (to produce ≧10 mEq of acid neutralizing capacity (ANC)).

Lansoprazole (≧1 mg) with antacid (to produce ≧10 mEq of acid neutralizing capacity (ANC)).

Rabeprazole (≧1 mg) with antacid (to produce ≧10 mEq of acid neutralizing capacity (ANC)).

Pantoprazole (≧1 mg) with antacid (to produce ≧10 mEq of acid neutralizing capacity (ANC)).

Esomeprazole (≧1 mg) with antacid (to produce ≧10 mEq of acid neutralizing capacity (ANC)).

Pariprazole (≧1 mg) with antacid (to produce ≧10 mEq of acid neutralizing capacity (ANC)).

Leminoprazole (≧1 mg) with antacid (to produce ≧10 mEq of acid neutralizing capacity (ANC)).

Omeprazole (≧1 mg) with antacid (to produce ≧20 mEq of acid neutralizing capacity (ANC)).

Lansoprazole (≧1 mg) with antacid (to produce ≧20 mEq of acid neutralizing capacity (ANC)).

Rabeprazole (≧1 mg) with antacid (to produce ≧20 mEq of acid neutralizing capacity (ANC)).

Pantoprazole (1 mg) with antacid (to produce ≧20 mEq of acid neutralizing capacity (ANC)).

Esomeprazole (≧1 mg) with antacid (to produce ≧20 mEq of acid neutralizing capacity (ANC)).

Pariprazole (≧1 mg) with antacid (to produce ≧20 mEq of acid neutralizing capacity (ANC)).

Leminoprazole (≧1 mg) with antacid (to produce ≧20 mEq of acid neutralizing capacity (ANC)).

As mentioned above the present invention can be prepared by any number of conventional dosage forming techniques known to those skilled in the art such as granulation, direct compression and/or capsule filling. In one embodiment of the present invention, the antacid and the proton pump inhibitor are separately granulated. The antacid granules will comprise at least the antacid and a binder. The proton pump inhibitor granules will comprise at least the proton pump inhibitor, a binder and an alkaline agent, preferably an alkaline amino acid. The granules may also comprise a filler, a disintegrant, a glidant, a lubricant and a taste masking agent. The granules can be made by wet or dry techniques commonly employed in the art. Slugging may also be employed to make the granules. In one embodiment of the present invention both the antacid granules and the proton pump inhibitor granules are prepared by a wet granulation technique. In another embodiment, the antacid granules and the proton pump inhibitor granules are made by dry granulation techniques such as roller compaction. In a further embodiment, the antacid granules are made by roller compaction and the proton pump inhibitor granules are made by wet granulation.

Because many of the proton pump inhibitors such as omeprazole elicit a bitter taste that is difficult to mask simply by the addition of sweeteners and flavoring agents, it may be necessary to coat or encapsulate the proton pump inhibitors with a film-forming polymer or a wax material especially if the dosage form of the present invention will be chewable or rapidly dissolving in the mouth. One particularly acceptable approach for coating the proton pump inhibitor involves melt granulation.

In melt granulation, a congealable solid, preferably a wax such as glyceryl monostearate or castor oil, is employed to coat or embed the proton pump inhibitor and thereby mask the bitter taste. The congealable solid must be non-toxic, stable with a low melting point and no interaction with the drug that will affect its bioavailability. The congealable solid is heated until it melts. The proton pump inhibitor and other excipients such as an alkaline material and a plasticizer are dispersed into melted material preferably by using a high-shear granulator with a temperature bath or control element that will prevent the congealable material from prematurely cooling and solidifying. After the proton pump and other excipients are dispersed in the melted congealable material the dispersion is allowed to cool and coated proton pump inhibitor granules are formed.

If a polymer coating of the proton pump inhibitor is selected, a fluidized bed or pan coater may be used to apply a polymer dispersion or solution onto a mixture of the proton pump inhibitor and selected excipients such as an alkaline material. The polymers used to coat and taste mask the proton pump inhibitor can be film forming water soluble or water insoluble polymers such as ethylcellulose, hydroxypropyl methylcellulose, hydroxypropyl cellulose or combinations of the foregoing. It is also possible to use a combination of congealable material and polymer to coat the proton pump inhibitor. In one embodiment of the present invention no acidic film forming polymers such as enteric polymer should be used. In an alternate embodiment enteric film forming polymers can be used.

Once the antacid granules and the proton pump inhibitor granules are prepared, they are then further mixed with additional excipients such as a taste masking agent, a glidant and a lubricant to form an antacid laying mixture and a proton pump layering mixture. The layering mixtures may also be mixed with additional fillers, binders and disintegrants. Depending upon the ingredients selected for the dosage formulation, the prior formation of antacid granules and proton pump inhibitor granules may not be necessary. If the materials selected for use in the antacid layering mixture and the proton pump inhibitor layering mixture allow sufficient flow of the mixtures into a tablet die or capsule without the need for a granulation step, the mixtures can be fed directly into a tablet press or capsule filing machine for the formation of the final dosage form.

After the antacid layering mixture and the proton pump inhibitor layering mixture have been prepared, with or with out the granulation step, the layering mixtures are then formed into the final dosage form. In one embodiment, a predetermined amount of the proton pump inhibitor layering mixture is fed into a tablet press to form the proton pump inhibitor layer then a predetermined amount of the antacid layering mixture is fed into the tablet press to form the antacid layer of the multi-layer tablet. It should be appreciated that the order in which the proton pump inhibitor layer and antacid layer are fed into the tablet press can be reversed. Additional antacid layers and proton pump inhibitor layers can also be fed into the tablet press. In one embodiment, the proton pump inhibitor layer is sandwiched between two antacid layers that contain the same or different antacids.

If a capsule is the final dosage form, a predetermined amount of the proton pump inhibiting layering mixture is fed into one half of a capsule. Once the proton pump inhibiting layering mixture is in the capsule, a predetermined amount of the antacid layering mixture is added to the capsule and forms an antacid layer on top of the proton pump inhibitor layer. Once both the proton pump inhibitor layer and the antacid layer are in the capsule, the capsule is sealed. Again the order in which the proton pump inhibitor layers are placed in the capsule can be reversed as well as the inclusion of additional layers. In an alternate capsule embodiment, a predetermined amount of the proton pump inhibitor layering mixture is placed into a small capsule and sealed. The small capsule is then placed into a larger capsule with a predetermined amount of the antacid layering mixture and the larger capsule sealed to form the multi-layer dosage formulation of the present invention. Again the order in which the proton pump inhibitor and antacid layering mixture is placed into the capsules can be reversed without depart from the scope of the present invention.

If a capsule is used in the final dosage form and the dosage form is designed to rapidly dissolve in the mouth, the capsule selected should be rapidly disintegrating. Such rapidly disintegrating capsules are commercially available from CAPSUGEL of Morris Plains, NJ under the tradename NPcaps™.

The phrase “predetermined amount” used above means an amount of layering mixture that is calculated to provide a therapeutic amount of the proton pump inhibitor (i.e. 5-200mg) and/or a therapeutic amount of antacid activity (i.e. 1-20 mEq).

The present invention also provides a method for treating a patient in need of therapy for gastrointestinal disorders. One embodiment of the method is as follows:

A method for treating gastrointestinal disorders comprising the steps of:

(a) combining a combination or single dosage form comprising a proton pump inhibitor in one distinct layer and an aluminum, magnesium or calcium antacid salt in another distinct layer into a chewable or rapidly dispersible dosage form comprising at least two layers; and

(b) providing said chewable or rapidly dispersible dosage form to a patient in need of therapy for gastrointestinal disorders.

If granules are employed in the present invention, the granules should comprise the following:

Proton Pump Inhibitor Granules:

| | Preferred | Most Preferred |
|---|---|---|
| Proton Pump Inhibitor | 5-60% | 10-40% |
| Alkaline Material | 5-60% | 10-40% |
| Filler | 10-90% | 20-80% |
| Binder | 0-50% | 1-30% |
| Disintegrant | 0-60% | 0-50% |
| Lubricant | 0-10% | 0-5% |
| Glidant | 0-10% | 0-5% |

Antacid Granules:

| | Preferred | Most Preferred |
|---|---|---|
| Antacid | 30-99% | 50-95% |
| Binder | 0.1-40% | 1-25% |
| Filler | 0-60% | 0-50% |
| Disintegrant | 0-60% | 0-50% |

The granules are further processed into distinct layering mixtures for tabletting or capsules as follows:

Proton Pump Inhibitor Layering Mixture

| | Preferred | Most Preferred |
|---|---|---|
| Proton Pump Granules | 40-99% | 50-95% |
| Taste Masking Agent* | 0-40% | 0-25% |
| Lubricant | 0-10% | 0-5% |
| Glidant | 0-10% | 0-5% |

*The taste masking agent preferably is a combination of a 0.1-99% sweetener and 0.1 to 99% favoring agent.

Antacid Layering Mixture

| | Preferred | Most Preferred |
|---|---|---|
| Antacid Granules | 40-99% | 50-95% |
| Taste Masking Agent* | 0-40% | 0-25% |
| Lubricant | 0-10% | 0-5% |
| Glidant | 0-10% | 0-5% |

*The taste masking agent preferably is a combination of a 0.1-99% sweetener and 0.1 to 99% favoring agent.

The layering mixtures are individually processed on a tablet press to produce a multi-layered (i.e. bilayer or trilayer) chewable tablet, or rapidly disintegrating tablets. The layering mixtures may also be individually processed into capsules. Whether the final dosage form is a tablet or capsule the antacid layer should comprises 40-95% of the final tablet weight, preferably, 50-85% and most preferably 60-80% and the proton pump inhibitor layer should comprises 5-60% of the final tablet weight, preferably, 15-50% and most preferably 20-40%. As mentioned above, the layering mixtures may not need the prior formation of granules. If the granules are not employed the layering mixtures may comprise above mentioned granule excipients in similar amount only in a non-granule form.

Several examples of embodiments that may be used to practice the subject invention are provided herein.

### EXAMPLE 1

A bilayer chewable tablet in accordance with the present invention was prepared as follows:

A batch of proton pump inhibitor granules was prepared using a top spray fluidized coater and the following ingredients:

| | |
|---|---|
| Omeprazole (non-micronized) | 180 g |
| L-Arginine | 180 g |
| Microcrystalline Cellulose (Avicel PH 102) | 450 g |
| Eudragit ® RD 100 | 90 g |
| Purified Water | q.s. (900 g) |

Eudragit RD was dissolved in water and L-Arginine and Omeprazole were evenly dispersed in the solution. Avicel PH 102 was loaded in the fluid bed coater and the solution was sprayed using below conditions:

Outlet temperature: 45±5 C.°

Inlet temperature: 70±5 C.°

Spraying rate: 5-6 ml/min

The water evaporates during the granulation and drying process.

A batch of antacid granules was prepared using a blender and the following ingredients.

| | |
|---|---|
| Aluminum Hydroxide | 95 g |
| Hydroxypropyl Methylcellulose (METHOCEL E5) | 5 g |
| Purified Water | q.s. (55.8 g) |

The resulting antacid granules were dried in an oven for approximately 24 hours at 80° C.

Some of the proton pump inhibitor granules prepared above were further processed into a proton pump layering mixture of the following composition:

| | |
|---|---|
| Omeprazole Granules | 5.39 g |
| Artificial Cherry Flavor | 0.108 g |
| Aspartame | 0.297 g |
| Colloidal Silicon Dioxide (CAB-O-SIL M5) | 0.108 g |
| Sodium Stearate | 0.405 g |

Some of the antacid granules prepared above were further processed into an antacid layering mixture of the following composition:

| | |
|---|---|
| Aluminum Hydroxide Granules | 22.719 g |
| Artificial Cherry Flavor | 0.108 g |
| Aspartame | 0.297 g |
| Sodium Stearate | 0.564 g |

The proton pump inhibitor layering mixture and the antacid layering mixture were individually processed on a tablet press using a 0.5″ flat face tooling and manually compressed to produce a bilayer chewable tablet wherein said antacid layer weighs 878.0 mg and said proton pump inhibitor layer weighs 234.0 mg.

### EXAMPLE 2

A bilayer chewable tablet in accordance with the present invention is prepared as follows

Some of the proton pump inhibitor granules prepared in Example 1 above were further processed into a proton pump layering mixture of the following composition:

| | |
|---|---|
| Omeprazole Granules | 4.615 g |
| Debittering Flavor (natural) | 0.069 g |
| Xylitol | 4.615 g |
| Colloidal Silicon Dioxide (CAB-O-SIL M5) | 0.092 g |
| Sodium Stearate | 0.392 g |

Some of the antacid granules prepared in Example 1 above were further processed into an antacid layering mixture of the following composition:

| | |
|---|---|
| Aluminum Hydroxide Granules | 19.433 g |
| Natural Mint Flavor | 0.087 g |
| Aspartame | 0.225 g |
| Sodium Stearate | 0.471 g |

The above proton pump inhibitor layering mixture and the antacid layering mixture were individually processed on a tablet press using a 0.5″ flat face tooling and manually compressed to produce a bilayer chewable tablet with said antacid layer weighing 876.0 mg and proton pump inhibitor layer weighing 424.0 mg.

### EXAMPLE 3

A bilayer chewable tablet in accordance with the present invention is prepared as follows

Some of the proton pump inhibitor granules prepared in Example 1 above were further processed into a proton pump layering mixture of the following composition:

| | |
|---|---|
| Omeprazole Granules | 4.580 g |
| Debittering Flavor (natural) | 0.115 g |
| Xylitol | 1.145 g |
| Mannitol | 3.435 g |
| Aspartame | 0.137 g |
| Colloidal Silicon Dioxide (CAB-O-SI, M5) | 0.092 g |
| Sodium Stearate | 0.389 g |

Some of the antacid granules prepared in Example 1 above were further processed into an antacid layering mixture of the following composition:

| | |
|---|---|
| Aluminum Hydroxide Granules | 19.285 g |
| Natural Mint Flavor | 0.135 g |
| Aspartame | 0.229 g |
| Sodium Stearate | 0.458 g |

The above proton pump inhibitor layering mixture and the antacid layering mixture were individually processed on a tablet press using a 0.5″ flat face tooling and manually compressed to produce a bilayer chewable tablet with said antacid layer weighing 878.0 mg and proton pump inhibitor layer weighing 200.0 mg.

### EXAMPLE 4

A trilayer chewable tablet in accordance with the present invention is prepared as follows

Some of the proton pump inhibitor granules prepared in Example 1 above were further processed into a proton pump layering mixture of the following composition:

| | |
|---|---|
| Omeprazole Granules | 4.478 g |
| Debittering Flavor (natural) | 0.134 g |
| Lactose Monohydrate (spray dried) | 4.478 g |
| Aspartame | 0.201 g |
| Colloidal Silicon Dioxide (CAB-O-SIL M5) | 0.090 g |
| Sodium Stearate | 0.358 g |

A batch of aluminum hydroxide antacid granules is prepared according to the procedure described in Example 1 above with the following ingredients:

| | |
|---|---|
| Aluminum Hydroxide | 90 g |
| Lactose Monohydrate | 5.0 g |
| Hydroxypropyl Methylcellulose (METHOCEL E5) | 5.0 g |
| Purified Water | q.s. (50.0 g) |

Some of the aluminum hydroxide antacid granules prepared above were further processed into an antacid layering mixture of the following composition:

| | |
|---|---|
| Aluminum Hydroxide Granules | 14.925 g |
| Natural Mint Flavor | 0.186 g |
| Aspartame | 0.224 g |
| Sodium Stearate | 0.448 g |

A calcium carbonate antacid layering mixture is prepared by blending the following ingredients:

| | |
|---|---|
| Calcium Carbonate | 2.69 g |
| Lactose Monohydrate (spray dried) | 1.34 g |
| Natural Mint Flavor | 0.070 g |
| Sodium Stearate | 0.380 g |

The above proton pump inhibitor layering mixture, the aluminum hydroxide antacid layering mixture, and the calcium carbonate antacid layering mixture were individually processed on a tablet press using a 0.5″ flat face tooling and manually compressed to produce a trilayer chewable tablet with said aluminum hydroxide antacid layer weighing 705 mg, proton pump inhibitor layer weighing 435 mg and the calcium carbonate antacid layer weighing 200 mg. The proton pump inhibitor layer was sandwiched between the two antacid layers.

### EXAMPLE 5

A bilayer orally disintegrating capsule in accordance with the present invention was prepared as follows:

A batch of proton pump inhibitor granules was prepared using a top spray fluidized coater and the following ingredients:

| | |
|---|---|
| Omeprazole (non-micronized) | 180 g |
| L-Arginine | 180 g |
| Microcrystalline Cellulose (Avicel PH 102) | 180 g |
| Lactose Monohydrate | 180 g |
| Eudragit ® RD 100 | 180 g |
| Purified Water | q.s. (1800 g) |

The water evaporates during the granulation and drying process.

A batch of antacid granules was prepared using a blender and the following ingredients.

| | |
|---|---|
| Aluminum Hydroxide | 95 g |
| Hydroxypropyl Methylcellulose (METHOCEL E5) | 5 g |
| Purified Water | q.s. (35.714 g) |

The resulting antacid granules were dried in an oven for approximately 24 hours at 80° C.

Some of the proton pump inhibitor granules prepared above were further processed into a proton pump layering mixture of the following composition:

| | |
|---|---|
| Omeprazole Granules | 4.580 g |
| Debittering Flavor (natural) | 0.115 g |
| Xylitol | 1.145 g |
| Mannitol | 3.435 g |
| Aspartame | 0.137 g |
| Colloidal Silicon Dioxide (CAB-O-SI, M5) | 0.092 g |
| Sodium Stearate | 0.389 g |

Some of the antacid granules prepared above were further processed into an antacid layering mixture of the following composition:

| | |
|---|---|
| Aluminum Hydroxide Granules | 19.285 g |
| Natural Mint Flavor | 0.135 g |
| Aspartame | 0.229 g |
| Sodium Stearate | 0.458 g |

Approximately 108 mg of the above proton pump inhibitor layering mixture was manually placed into a rapidly disintegrating size “0” capsule, commercially available from Capsugel under the tradename NPcapS™. After the proton pump inhibitor layering mixture was placed in the capsule, approximately 219.5 mg of the antacid layering mixture was manually placed into the same rapidly disintegrating capsule thereby forming a proton pump inhibitor layer and an antacid layer within the rapidly disintegrating capsule. Once both components have been placed in the capsule, the capsule is sealed. When the filled capsule is placed in the mouth of a human, it disintegrated in about 10-20 seconds, without the need for additional water, and released it contents with no bitter taste or components adhering to the teeth and gums.

### EXAMPLE 6

A bilayer orally disintegrating capsule in accordance with the present invention was prepared as follows:

Approximately 108 mg of the proton pump inhibitor layering mixture from Example 5 above was manually placed into a size “4” rapidly disintegrating capsule and sealed. The sealed capsule was then placed inside a size “00” rapidly disintegrating capsule along with approximately 219.5 mg of the antacid layering mixture from Example 5 above and sealed. The dual capsule is then placed in the mouth of a human where the first capsule disintegrated within 10-20 seconds and the internal capsule disintegrated within 15 seconds without the need for additional water. The contents of both capsules were released into the patient's mouth without a bitter taste or adhesion of the contents to the teeth and gums.

### EXAMPLE 7

An aluminum hydroxide layering mixture and a magnesium carbonate layering mixture in accordance with the present invention, but without the need of preparing the antacid granules prior to preparation of the layering mixture, were prepared as follows:

The following ingredients were sieved and manually mixed in a plastic bag then fed into a roller compactor for a dry compaction-granulation process.

Aluminum Hydroxide Blend

| | % w/w | mg/tablet | g/batch |
|---|---|---|---|
| Dried Aluminum Hydroxide Gel, USP | 95.0 | 475.0 | 237.5 |
| Microcrystalline Cellulose, NF | 3.0 | 15.0 | 7.50 |
| (Avicel PH 101) | | | |
| Aspartame, NF | 0.5 | 2.5 | 1.25 |
| Colloidal Silicon Dioxide, NF | 0.5 | 2.5 | 1.25 |
| (Cab-O-Sil, M-5) | | | |
| Magnesium Stearate*, NF | 1.0 | 5.0 | 2.5 |
| Total | 100.0 | 500.0 | 250.0 |

*Half of the amount of magnesium stearate was added to the blend prior to roller compaction, the remaining half of the magnesium stearate was added to the resulting granules after roller compaction and milling.

Magnesium Carbonate Blend

| | % w/w | mg/tablet | g/batch |
|---|---|---|---|
| Magnesium Carbonate, USP | 93.0 | 604.5 | 302.25 |
| (heavy powder) | | | |
| Microcrystalline Cellulose, NF | 5.0 | 32.5 | 16.25 |
| (Avicel PH 101) | | | |
| Aspartame, NF | 0.5 | 3.25 | 1.625 |
| Colloidal Silicon Dioxide, NF | 0.5 | 3.25 | 1.625 |
| (Cab-O-Sil, M-5) | | | |
| Magnesium Stearate*, NF | 1.0 | 6.50 | 3.25 |
| Total | 100.0 | 650.0 | 325.0 |

*Half of the amount of magnesium stearate was added to the blend prior to roller compaction, the remaining half of the magnesium stearate was added to the resulting granules after roller compaction and milling.

Each of the above blends were separately fed into a TF mini roller compactor, using a roller speed of 5 rpms, feed screw speed of 15 rpms and applied pressure of 2 tons. The resulting sheets were fed through a comil equipped with a No. 1397 screen and blade speed of 690 rpms. The bulk and tap density of the milled granules was determined and compared to the dry blend. The results are reported in table 1.

TABLE 1

| | | |
|---|---|---|
| Bulk | | |
| Density | Tap Density | Compressibility |
| (g/cc) | (g/cc) | (%) |

DRY BLEND

| | | | |
|---|---|---|---|
| Magnesium Carbonate Blend | 0.36 | 0.55 | 52.8 |
| Aluminum Hydroxide Blend | 0.28 | 0.43 | 53.8 |

AFTER ROLLER COMPACTION

| | | | |
|---|---|---|---|
| Magnesium Carbonate Blend | 0.63 | 0.85 | 34.9 |
| Aluminum Hydroxide Blend | 0.51 | 0.71 | 39.2 |

The compressibility was determined according to the following formula:

(tap density−bulk density)/bulk density×100.

As shown by the above data, the bulk density and tap density increased significantly after roller compaction and the compressibility also improved, suggesting increased flowability and a reduction in the cohesiveness of the particles.

The particle size of the roller compacted material was also determined. The results of the particle size analysis are reported in Table 2.

TABLE 2
% RETAINED

| Mesh Size | Magnesium Carbonate | Aluminum Hydroxide |
|---|---|---|
| 20 | 34.9 | 40.9 |
| 40 | 40.6 | 27.7 |
| 60 | 11.2 | 8.4 |
| 80 | 4.2 | 12.0 |
| 100 | 2.4 | 5.6 |
| Pan | 6.7 | 5.4 |
| Total | 100 | 100 |

The aluminum hydroxide blend and the magnesium carbonate blends after roller compaction and milling can be used to as an antacid layering mixture in Examples 1-5 above.

### EXAMPLE 8

A taste masked proton pump inhibitor granule for use in the present invention was prepared as follows:

Approximately 300 g of glyceryl monostearate are placed in a high shear granulator and heated to approximately 60° C. Once the glyceryl monostearate has melted, approximetly 75-80 g of omerpazole, 75 g of arginine, and 45-50 g of polyethylene glycol 3350 are added to the granulation bowl and mixed until the omeprazole is dispersed throughout the glyceryl monstearate. The mixture in the granulation bowl is cooled producing proton pump inhibitor granules that can be used to make proton pump inhibitor layering mixtures and multi-layered tablets and capsules as described in Examples 1-6 above. Other grades of polyethylene glycol (PEG) could also be used in this formulation such as PEG 1450. The selection of the grade and amount of PEG is within the skill of the ordinary formulator.

### EXAMPLE 9

A taste masked proton pump inhibitor formulation in accordance with the present invention was prepared as follows:

A granulation solution is prepared by dissolving about 60 g of povidone K-30 in about 160 g of purified water.

About 120 g of meglumine, 125 g of omeprazole, 65 g of hypromellose 2208 and 630 g of mannitol are blended in a Diosner mini granulator and granulated with the previously prepared granulation solution. The granules are dried in a heated oven for about 4 hours at a temperature of 60 ±5° C. The dried granulation is milled at low speed through a comil using a #1397 screen to obtain omeprazole granules.

About 36.25 g of hydroxypropyl cellulose (KUCEL 9410 HXF), 6.0 g of microcrystalline cellulose (AVICEL PH 101), 1.25 g of aspartame, 0.625 g of colloidal silicon dioxide (CAB—O—SIL M-5) all of which were previously passed through a #25 mesh screen, were added to the dried omeprazole granules and blended for about 10 minutes. About 0.875 g of magnesium stearate that had been passed through a #25 mesh screen was added to the blended omeprazole granules and blended for an additional three minutes.

The blended omeprazole granules were then processed in a tablet press with the aluminum hydroxide antacid layering mixture and the calcium carbonate antacid layering mixture from Example 7above using a 0.5″ flat face compound cup tooling and manually compressing to produce a trilayer layer tablet with the aluminum hydroxide layer weighing 500 mg, the proton pump inhibitor layer weighing 250 mg and the magnesium carbonate layer weighing 650 mg. The proton pump inhibitor layer was sandwiched between the two antacid layers.

### EXAMPLE 10

A taste masked proton pump inhibitor formulation in accordance with the present invention was prepared as follows:

A granulation solution is prepared by dissolving about 65 g of povidone K-30 in about 130 g of purified water.

About 150 g of arginine, 150 g of micronized omeprazole, and 635 g of mannitol are blended in a Diosner mini granulator and granulated with the previously prepared granulation solution. The granules are dried in a heated oven for about 2 hours at a temperature of 60 ±5° C. The dried granulation is milled at low speed through a comil using a #1397 screen to obtain omeprazole granules.

About 32.08 g of glyceryl monostearate, 15.0 g of hypromellose 2910 and 6.25 g of polyethylene glycol 3350, previously passed through a #25 mesh screen were added to the dried omeprazole granules and blended for about 10 minutes.

The blended omerpazole granules were then processed in a tablet press with the aluminum hydroxide antacid layering mixture and the calcium carbonate antacid layering mixture from Example 7 above using a 0.5″ flat face compound cup tooling and manually compressing to produce a trilayer layer tablet with the aluminum hydroxide layer weighing 500 mg, the proton pump inhibitor layer weighing 250 mg and the magnesium carbonate layer weighing 650 mg. The proton pump inhibitor layer was sandwiched between the two antacid layers.

### EXAMPLE 11

A taste masked proton pump inhibitor granule for use in the present invention was prepared as follows:

Approximately 10 g of the omeprazole granules prepared in Example 9 above were then coated with a coating solution containing:

10.6 g of ethylcellulose (ETHOCEL® 7 cps)

42.4 g of hydroxypropyl methylcellulose (METHOCEL® E6LV)and

980 g of 80% ethanol.

The coating solution was applied using a fluid-bed coater (UniGlatt) by side spraying under the following conditions:

| | |
|---|---|
| Outlet temperature: | 35-40° C. |
| Inlet temperature: | 65-75° C. |
| Solution Feed Rate: | 5 mL/min |

The resulting coated omeprazole granules can be used to make proton pump inhibitor layering mixtures in multi-layered tablets and capsules as described in the Examples above.

### EXAMPLE 12

A taste masked proton pump inhibitor granule for use in the present invention was prepared as follows:

Approximately 100 g of the omeprazole granules prepared in Example 9 above were then coated with a coating solution containing about 47-53 g of hydroxypropyl methylcellulose (METHOCEL® E6LV) in about 980 g of purified water.

The coating solution was applied using a fluid-bed coater using the conditions described in Example 11.

The resulting coated omeprazole granules can be used to make proton pump inhibitor layering mixtures in multi-layered tablets and capsules as described in Examples above.

### EXAMPLE 13

A taste masked proton pump inhibitor granule for use in the present invention was prepared as follows:

Approximately 100 g of the omeprazole granules prepared in Example 9 above were then coated with a coating solution containing 45-53 g of hydroxypropyl cellulose (KLUCEL® EF) in about 980 g of purified water.

The coating solution was applied using a fluid-bed coater using the conditions described in Example 11.

The resulting coated omeprazole granules can be used to make proton pump inhibitor layering mixtures in multi-layered tablets and capsules as described in Examples above.

### EXAMPLE 14

A taste masked proton pump inhibitor granule for use in the present invention was prepared as follows:

Approximately 100 g of the omeprazole granules prepared in Example 9 above were then coated with a coating suspension containing:

10 g of ethylcellulose (ETHOCEL® 7 cps)

60 g of glyceryl monostearate and

200 g of 80% ethanol

The ethylcellulose was dissolved in ethanol and the glyceryl monostearate was dispersed in the ethylcellulose/ethanol solution to create the coating suspension

The coating suspension was applied using a fluid-bed coater using the conditions described in Example 11.

The resulting coated omeprazole granules can be used to make proton pump inhibitor layering mixtures in multi-layered tablets and capsules as described in Examples above.

### EXAMPLE 15

A taste masked proton pump inhibitor granule for use in the present invention was prepared as follows:

Approximately 100 g of the omeprazole granules prepared in Example 9 above were then coated with coating solution containing 6.5 g of hydroxypropyl cellulose (KLUCEL® EF), 58.5 g of glyceryl monostearate and 130 g of purified water.

The coating solution was applied using a fluid-bed coater using the conditions described in Example 11.

The resulting coated omeprazole granules can be used to make proton pump inhibitor layering mixtures in multi-layered tablets and capsules as described in Examples above.

### EXAMPLE 16

A taste masked proton pump inhibitor granule for use in the present invention was prepared as follows:

10 Omeprazole granules were prepared as follows:

30% omeprazole, 10% ethylcellulose (ETHOCEL 7cps) and 60% glyceryl monostearate were dispersed in 80% ethanol to make 20 w/w% suspension. After the suspension was homogenized, it was spray dried using a fluid-bed coater (UniGlatt) by side spraying under the following conditions:

| | |
|---|---|
| Outlet temperature: | 35-40° C. |
| Inlet temperature: | 65-75° C. |
| Solution Feed Rate: | 5 mL/min |

The resulting coated omeprazole granules can be used to make proton pump inhibitor layering mixtures in multi-layered tablets and capsules as described in Examples above.

### EXAMPLE 17

A taste masked proton pump inhibitor granule for use in the present invention was prepared as follows:

Omeprazole granules were prepared as follows:

35% omeprazole, 60% glyceryl monostearate and 5% of a surfactant (either polyethylene glycol [PEG 400] or poloxamer [Pluronic F-68]) were dispersed in 80% ethanol to make 20 w/w% suspension. After the suspension was homogenized, it was spray dried using a fluid-bed coater (UniGlatt) by side spraying under the following conditions:

| | |
|---|---|
| Outlet temperature: | 35-40° C. |
| Inlet temperature: | 65-75° C. |
| Solution Feed Rate: | 5 mL/min |

The resulting coated omeprazole granules can be used to make proton pump inhibitor layering mixtures in multi-layered tablets and capsules as described in Examples above.

### EXAMPLE 18

A taste masked proton pump inhibitor granule for use in the present invention was prepared as follows:

Approximately 67 g of glyceryl monostearate, 30 g of micronized omeprazole, 30 g of arginine, 44 g of mannitol, 4 g NaCl and 12 g of ethylcellulose (ETHOCEL® 7cps) in 35 g of 80% ethanol as a binder solution was placed in a high shear granulator (VG-5) and mixed for approximately 10 minutes. After drying, the resulting proton pump inhibitor granules can be used to make proton pump inhibitor layering mixtures in multi-layered tablets and capsules as described in Examples above.

While certain preferred and alternative embodiments of the invention have been set forth for purposes of disclosing the invention, modifications to the disclosed embodiments may occur to those who are skilled in the art. Accordingly, the appended claims are intended to cover all embodiments of the invention and modifications thereof that do not depart from the spirit and scope of the invention.

What is claimed is:

1. A multi-layered pharmaceutical dosage form comprising:
a. at least one proton pump inhibitor layer comprising a proton pump inhibitor, an alkaline agent, and an additional pharmaceutical excipient wherein the proton pump inhibitor layer is free of enteric coating; and
b. at least one antacid layer comprising an aluminum, magnesium or calcium antacid salt and a pharmaceutically acceptable excipient, wherein the proton pump inhibitor layer and the antacid layer are distinct from each other.

2. The pharmaceutical dosage form as defined in claim 1 that comprises at least two antacid layers.

3. The pharmaceutical dosage form as defined in claim 1 wherein the dosage form is a compressed tablet.

4. The pharmaceutical dosage form as defined in claim 1 wherein the dosage form is a capsule.

5. The pharmaceutical dosage form as defined in claim 3 wherein the tablet is chewable.

6. The pharmaceutical dosage form as defined in claim 3 wherein the tablet is rapidly disintegrating.

7. The pharmaceutical dosage form as defined in claim 4 wherein the capsule is rapidly disintegrating.

8. The pharmaceutical dosage form as defined in claim 1 wherein the alkaline agent is an alkaline amino compound.

9. The pharmaceutical dosage form as defined in claim 8 wherein the alkaline agent is arginine or lysine.

10. The pharmaceutical dosage form as defined in claim 1 wherein the proton pump inhibitor is omeprazole, lansoprazole, pantoprazole, pariprazole, leminoprazole, salts, isomers, or derivatives thereof.

11. The pharmaceutical dosage form as defined in claim 1 wherein the antacid salts are aluminum or calcium salts of hydroxides, carbonates, sulfates, bicarbonates, or silicates.

12. The pharmaceutical dosage form as defined in claim 11 wherein the antacid salts are aluminum or calcium salts of hydroxides, carbonates, sulfates, or silicates.

13. The pharmaceutical dosage form as defined in claim 1 wherein the proton pump inhibitor is coated with a film-forming polymer or congealable solid material.

14. The pharmaceutical dosage form as defined in claim 13 wherein the film-forming polymer comprises a water soluble polymer, a water insoluble polymer or a combination of a water insoluble polymer and a water soluble polymer.

15. The pharmaceutical dosage form as defined in claim 13 wherein the congealable solid material is a wax.

16. The pharmaceutical dosage form as defined in claim 13 wherein the congealable solid material is glyceryl monostearate or castor oil.

17. The pharmaceutical dosage form as defined in claim 13 wherein the proton pump inhibitor is coated with a combination comprising a film-forming polymer and a congealable solid material.

18. The pharmaceutical dosage form as defined in claim 1 wherein the proton pump inhibitor layer further comprises: (a) granules that comprise a proton pump inhibitor, an alkaline agent and a binder and (b) a taste masking agent.

19. The pharmaceutical dosage form as defined in claim 1 wherein the antacid layer further comprises granules that comprise an antacid and a binder.

20. The pharmaceutical dosage form as defined in claim 19 wherein the antacid layer further comprises a taste masking agent.

21. The pharmaceutical dosage form as defined in claim 19 wherein the granules are prepared by a dry granulation technique.

22. The pharmaceutical dosage form as defined in claim 22 wherein the granules are prepared by roller compaction.

23. The pharmaceutical dosage form as defined in claim 19 wherein the granules are prepared by a wet granulation technique.

24. A method for preparing a multi-layered pharmaceutical tablet dosage formulation comprising the steps of:
(a) preparing a proton pump inhibitor layering mixture comprising a proton pump inhibitor, an alkaline agent and a taste masking agent;
(b) preparing an antacid layering mixture comprising an aluminum, magnesium or calcium antacid salt, at least one pharmaceutically acceptable excipient and a taste masking agent;
(c) feeding the proton pump inhibitor layering mixture into a tablet die to create at least one proton pump inhibitor layer;
(d) feeding the antacid layering mixture into a tablet die to create at least one antacid layer;
(e) combining the proton pump inhibitor layer and antacid layer to form a single unitary tablet with separate and distinct layers that contain at least one proton pump inhibitor layer and at least one antacid layer and wherein the tablet is free of enteric coatings.

25. A method for preparing a multi-layered pharmaceutical capsule dosage formulation comprising the steps of:
(f) preparing a proton pump inhibitor layering mixture comprising a proton pump inhibitor, an alkaline agent and a taste masking agent;
(g) preparing an antacid layering mixture comprising an aluminum, magnesium or calcium antacid salt, at least one pharmaceutically acceptable excipient and a taste masking agent;
(h) feeding the proton pump inhibitor layering mixture into a capsule shell to create at least one proton pump inhibitor layer;
(i) feeding the antacid layering mixture into a capsule shell to create at least one antacid layer to form a single unitary capsule with separate and distinct layers that contain a proton pump inhibitor and an antacid wherein the capsule is free of enteric coatings.

26. A method for providing concurrent therapy of gastrointestinal disorders comprising the steps of:
a. preparing a dosage form as defined in claim 1; and
b. administering the dosage form to a patient in need of therapy for gastrointestinal disorders.

27. The method as defined in claim 26 wherein the dosage form is chewable.

28. The method as defined in claim 26 wherein the dosage form is rapidly disintegrating.
